(19) United States
(12) Patent Application Publication
(10) Pub. No.: US 2010/0255584 A1
(43) Pub. Date: Oct. 7, 2010

(54) Arabidopsis thaliana Genome Sequence and Uses Thereof

(75) Inventors: Cao YONGWEI, Lexington, MA (US); David KOVALIC, University City, MO (US); Jingdong LIU, Ballwin, MO (US); James MCININCH, Burlington, MA (US); Steven ROUNSLEY, Melrose, MA (US); Sai SUBRAMANIAM, Framingham, MA (US); Roger WIEGAND, Wayland, MA (US)

Correspondence Address: ARNOLD & PORTER LLP, 555 TWELFTH STREET, N.W., ATTN: IP DOCKETING, WASHINGTON, DC, 20004, UNITED STATES, 202-942-5000, IP.Docketing@aporter.com

(73) Assignee: MONSANTO TECHNOLOGY LLC, St. Louis, MO (US)

(21) Appl. No.: 09/474,435

(22) Filed: Dec. 28, 1999

Related U.S. Application Data
Application No. 09/474,435 is a continuation-in-part of application No. 09/459,109, filed on Dec. 13, 1999, now abandoned.
Application No. 09/474,435 is a continuation-in-part of application No. 09/459,110, filed on Dec. 14, 1998, now abandoned.
(60) Provisional application No. 60/114,151, filed on Dec. 29, 1998.
(60) Provisional application No. 60/120,644, filed on Feb. 18, 1999.
(60) Provisional application No. 60/135,825, filed on May 24, 1999.
(60) Provisional application No. 60/139,932, filed on Jun. 21, 1999.
(60) Provisional application No. 60/143,994, filed on Jul. 15, 1999.
(60) Provisional application No. 60/155,422, filed on Sep. 23, 1999.
(60) Provisional application No. 60/111,990, filed on Dec. 14, 1998.
(60) Provisional application No. 60/111,991, filed on Dec. 14, 1998.

(51) Int. Cl. C12N 5/10; C07H 21/04

(52) U.S. Cl. 435/419; 536/23.1

(57) ABSTRACT

The present invention relates to nucleic acid sequences from the dicotyldonus plant Arabidopsis thaliana and, in particular, to genomic DNA sequences. The invention encompasses nucleic acid molecules present in non-coding regions as well as nucleic acid molecules that encode proteins and fragments of proteins. In addition, proteins and fragments of proteins so encoded and antibodies capable of binding the proteins are encompassed by the present invention. The invention also encompasses oligonucleotides including primers, e.g. useful for amplifying nucleic acid molecules, and collections of nucleic acid molecules and oligonucleotides, e.g. in microarrays. The invention also provides constructs and transgenic cells and organisms comprising nucleic acid molecules of the invention. The invention also relates to methods of using the disclosed nucleic acid molecules, oligonucleotides, proteins, fragments of proteins, and antibodies, for example, for gene identification and analysis, and preparation of constructs and transgenic cells and organisms.

This application claims priority under 35 U.S.C. §119(e) of U.S. Provisional Applications Nos. 60/089,524, filed Jun. 16, 1998; 60/089,516, filed Jun. 16, 1998; 60/089,808, filed Jun. 18, 1998; 60/089,812, filed Jun. 18, 1998; 60/089,807, filed Jun. 18, 1998; 60/089,806, filed Jun. 18, 1998; 60/089,811, filed Jun. 18, 1998; 60/089,813, filed Jun. 18, 1998; 60/111,990, filed Dec. 14, 1998; 60/111,991, filed Dec. 14, 1998; 60/114,151, filed Dec. 29, 1998; 60/120,644, filed Feb. 18, 1999; 60/135,825, filed May 24, 1999; 60/139,932, filed Jun. 21, 1999; 60/143,994, filed Jul. 15, 1999; and 60/155,422, filed Sep. 23, 1999; and under 35 U.S.C. §120 of U.S. application Ser. No. 09/333,534, filed Jun. 14, 1999; 09/459,109, filed Dec. 13, 1999; and 09/459,110, filed Dec. 13, 1999, the disclosures of which applications are incorporated herein by reference in their entirety.

The entirety of the Sequence Listing of U.S. Provisional Patent Application Ser. No. 60/114,151, filed on Dec. 29, 1998, is herein incorporated by reference.

### FIELD OF THE INVENTION

Included in the disclosure are nucleic acid molecules representing the genome of the dicotyledonous plant Arabidopsis thaliana and, in particular, to nucleic acid molecules having nucleic acid sequences corresponding to genes, promoters, other regulatory elements, and introns found in the Arabidopsis, thaliana genome, a specific set of genes of Arabidopsis thaliana and a set of primers based on the Arabidopsis thaliana genes. Also disclosed are homologous nucleic acid molecules, complementary nucleic acid molecules, polypeptides expressed by such genes, constructs comprising such promoters, regulatory elements and/or genes, transformed cells and organisms comprising such genes and/or promoters and regulatory elements, primers useful for replicating parts of such genes and nucleic acid molecules, computer readable media comprising sets of such nucleic acid sequences, polypeptides and primers, collections of nucleic acid molecules and methods of using such molecules and sequences including the use of collections of nucleic acid molecules in genetic research and clinical analysis, e.g. for gene expression.

### BACKGROUND OF THE INVENTION

Arabidopsis thaliana is a small plant in the mustard family which is widely studied as a model organism for plants in general, dicotyledonous plants in particular and most especially for plants in the Brassicaceae family including oil seed rape (canola), cabbage, cauliflower, broccoli, kohlrabi, turnips, Brussel Sprouts, radish and watercress. Arabidopsis, thaliana is a small, compact, prolific-seed producing plant with a short life cycle. The genome is believed to comprise about 20,000 to 30,000 genes on five chromosomes.

This dicotyledonous plant has been employed in investigations into a variety of genetic phenomena including the metabolic pathway functions, stress response and general cell development. A set of nucleic acid molecules representing a substantial set of the genes in the Arabidopsis, thaliana genome is useful in transcription profiling work to find, identify and characterize counterpart genes in other species, particularly plants and especially in dicotyledonous plant species. For instance, it is possible to identify unknown plant gene function by studying a similar (homologous) gene in a model plant in which genetic modification can more easily be done. That is, if unknown genes are disrupted or overexpressed, transcription profiling can be carried out to understand effects of the genetic modification.

Moreover, chemical discovery for plant treatment/regulation can be practiced using such transcription profiling with nucleic acids molecules of the Arabidopsis thaliana genome. In addition environmental stress studies of the Arabidopsis, thaliana genome will provide insight into related mechanisms in plants, e.g. yield, stability, thermal resistance, water/drought tolerance, etc.

Nucleic acid sequences of a species, e.g. the Arabidopsis thaliana can be generated by random shotgun sequencing of cloned genomic DNA and assembled into longer lengths of contiguous sequence (contigs). The final data set from an assembly process comprises a collection of sequences, which includes the contigs resulting from linking of two or more overlapping sequences as well as singleton nucleic acid sequences, i.e. trace sequences which are not incorporated into contigs. Such sequences can be screened for genes, e.g. full length or substantially full length or partial length genes. Screening methods include homology searches against databases of known genes and predictive methods using algorithms which infer the presence and extent of a gene.

The nucleic acid sequences disclosed herein are believed to represent a substantial number, or at least a major part, of the genes in the Arabidopsis thaliana genome. Genome sequence information from Arabidopsis thaliana permits identification of genetic sequences from other organisms including plants, animals (e.g. mammals such as humans), bacteria and fungi by comparison of such sequences with Arabidopsis thaliana sequences. The availability of a substantial set of genes or partial genes of the Arabidopsis thaliana genome permits the definition of primers for fabricating representative nucleic acid molecules of the genome which can be used on microarrays facilitating transcription profile studies. In addition the identification of the Arabidopsis thaliana genome permits the fabrication of a wide variety of DNA constructs useful for imparting unique genetic properties into transgenic organisms. These and other advantages attendant with the various aspects of this invention will be apparent from the following description of the invention and its various embodiments.

### SUMMARY OF THE INVENTION

The present invention contemplates and provides a substantial part of the genome of the dicotyledonous plant Arabidopsis thaliana. One aspect of the invention is a set of more than 81,000 contig and singleton sequences comprising coding sequence as well as promoters, other regulatory elements and introns identified from Arabidopsis thaliana ecotype Landsberg erecta and represented by SEQ ID NO: 1 through SEQ ID NO: 81306 in Table 1. Contigs in SEQ ID NO: 1 through SEQ ID NO: 81306 are recognized as those sequences whose designations begin with ATL8C; singleton sequences are recognized as those having designations which begin with ATL8S. Thus, a subset of the nucleic acid molecules of this invention comprises promoters and/or other regulatory elements of the Arabidopsis, thaliana genome as present in SEQ ID NO: 1 through SEQ ID NO: 81306 or complements thereof.

Another aspect of this invention is a set of clustered ESTs which are derived from various tissues of Arabidopsis thaliana ecotype Columbia. The ESTs represent consensus contiguous sequence (contig) and EST sequences which were not included within a contig as present in SEQ ID NO: 81307 through SEQ ID NO: 138060 in Table 2.

Another aspect of this invention comprises a set of more than 57,000 genes or partial genes of the Arabidopsis thaliana genome including genes represented by SEQ ID NO: 138061 through SEQ ID NO: 195836. As used herein, a substantial large set of genes for an organism is referred to as a unigene set. Thus, as used herein reference is made to specific genes comprising the unigene set of Arabidopsis thaliana as “ATUxxxxx” where ATU is an acronym for Arabidopsis thaliana unigene and xxxxx represents a number. Thus, ATU00001 to ATU057776 are used to designate the genes of Arabidopsis thaliana identified herein which are expected to include previously reported genes of Arabidopsis thaliana. Moreover, the term “ATU” by itself is also used herein to mean any of the nucleic acid molecules comprising genes or partial genes of the unigene set for Arabidopsis thaliana. More particularly the term “ATU of this invention” as used herein means a nucleic acid molecule representing a gene or partial gene of Arabidopsis thaliana disclosed herein selected from the group consisting of ATU00001 to ATU057776.

The present invention also contemplates and provides substantially purified nucleic acid molecules comprising the ATUs and other nucleic acid molecules of this invention as well as molecules which are complementary to, and capable of specifically hybridizing to, the ATU or its complement.

The present invention also contemplates and provides substantially purified nucleic acids molecules which are homologous to the nucleic acid molecules of this invention including, for example, those which are homologous to the ATUs of this invention, e.g. a plurality of related sets of homologous nucleic acid molecules in other species which are homologous to the ATUs.

The present invention also contemplates and provides substantially purified protein, or polypeptide fragments thereof, which are encoded by cDNA associated with the ATUs of the present invention.

The present invention also contemplates and provides constructs comprising promoters, regulatory elements and/or the ATUs which are useful in making transgenic cells or organisms. In particular this invention also provides transformed cell or organism having a nucleic acid molecule which comprises: (a) a promoter region which functions in the cell to cause the production of a mRNA molecule; which is linked to (b) a structural nucleic acid molecule, which is linked to (c) a 3′ non-translated sequence that functions in the cell to cause termination of transcription and addition of polyadenylated ribonucleotides to a 3′ end of the mRNA molecule, where components (a) and/or (b) are selected from Arabidopsis thaliana nucleic acid sequences provided herein and more preferably selected Arabidopsis thaliana nucleic acid sequences from the group consisting of ATU00001 to ATU057776.

Still another aspect of this invention is a set (and subsets thereof) of primers for the ATUs of this invention, which can be used to generate and isolate nucleic acid molecules representative ATUs of this invention and homologs thereof in other non-Arabidopsis thaliana species. The nucleic acids molecules of this invention including primers represent a useful tool in genetic research not only for the species Arabidopsis thaliana, but also for other plant species, both monocotyledonous as well as dicotyledonous, and other microorganisms and life forms with more differentiated cell structure such as plants and animals. The present invention also contemplates and provides primer pairs for replicating or identifying parts of the ATUs.

The present invention also contemplates and provides computer readable media having recorded thereon one or more of the nucleotide sequences provided by this invention and methods for using such media, e.g. in searching to identify genes associated with nucleic acid sequences.

The present invention also contemplates and provides collections of nucleic acid molecules, including oligonucleotides, representing the Arabidopsis thaliana genome including collections on solid substrates, e.g. substrates having attached thereto in array form nucleic acid molecules or oligonucleotides representing genes of the Arabidopsis thaliana genome. The invention also contemplates and provides methods of using such collections and arrays, e.g. in transcription profiling analysis. The present invention also contemplates and provides methods for using the nucleic acid molecules of this invention, e.g. for identifying genetic material and/or determining gene expression by hybridizing expressed and labeled nucleic acid molecules or fragments thereof to arrayed collections of the nucleic acid molecules of this invention.

The present invention also contemplates and provides oligonucleotides which are identical or complementary to a sequence of similar length for an ATU. Such oligonucleotides are useful, for example, for hybridizing to and identifying nucleic acid molecules which are homologous and/or complementary to the ATUs of the present invention.

The present invention also contemplates and provides methods for determining gene expression comprising collecting mRNA from tissue of an organism, using the mRNA as a template for producing a quantity of a labeled nucleic acid molecule, and contacting the labeled nucleic acid molecule with a collection of purified nucleic acid molecules of this invention, e.g. deposited in an array on a substrate.

### DETAILED DESCRIPTION OF THE INVENTION

As used herein, a nucleic acid molecule and/or polypeptide molecule, be it a naturally occurring molecule or otherwise, may be “substantially purified”, if the molecule is separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The ATUs of this invention and other nucleic acid molecules and/or polypeptide molecules of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

As used herein the term “polypeptide” means a protein or fragment thereof expressed by a nucleic acid molecule in a cell.

The ATUs of this invention and other nucleic acid molecules of the present invention may also be recombinant. As used herein, the term recombinant means any molecule (e.g. DNA, peptide etc.), that is, or results, however indirect, from human manipulation of a nucleic acid molecule.

It is understood that the nucleic acid molecules of the present invention may be labeled with reagents that facilitate detection of the agent, e.g. fluorescent labels as disclosed in U.S. Pat. No. 4,653,417, chemical labels as disclosed in U.S. Pat. Nos. 4,582,789 and 4,563,417 and modified bases as disclosed in U.S. Pat. No. 4,605,735, all of which are incorporated herein by reference in their entirety.

The term “oligonucleotide” as used herein refers to short nucleic acid molecules useful, e.g. for hybridizing probes, nucleotide array elements or amplification primers. Oligonucletide molecules are comprised of two or more nucleotides, i.e. deoxyribonucleotides or ribonucleotides, preferably more than five and up to 30 or more. The exact size will depend on many factors, which in turn depend on the ultimate function or use of the oligonucleotide. Oligonucleotides can comprise ligated natural nucleic molecules acids or synthesized nucleic acid molecules and comprise between 5 to 150 nucleotides or between about 15 and about 100 nucleotides, or preferably up to 100 nucleotides, and even more preferably between 15 to 30 nucleotides or most preferably between 18-25 nucleotides, identical or complementary to a sequence of similar length for an ATU.

This invention provides oligonucleotides specific for ATU sequences. Such oligonucleotides may be nucleic acid elements for use on solid arrays (e.g. synthesized or spotted) or primers for amplification of ATUs of this invention. Such primers for use in polymerase chain reaction (PCR) primers are preferably designed with the goal of amplifying nucleic acids from either the 3′ or the 5′ end of an ATU or a fragment of an ATU, e.g. about 500 to 800 bp of nucleic acids from the at the 3′ end of such a nucleic acid molecule.

The term “primer” as used herein refers to a nucleic acid molecule, preferably an oligonucleotide whether derived from a naturally occurring molecule, such as one isolated from a restriction digest or one produced synthetically, which is capable of acting as a point of initiation of synthesis when placed under conditions in which synthesis of a primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains at least 15, more preferably 18 nucleotides, which are identical or complementary to the template and optionally a tail of variable length which need not match the template. The length of the tail should not be so long that it interferes with the recognition of the template. Short primer molecules generally require cooler temperatures to form sufficiently stable hybrid complexes with the template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer. Computer generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123 (1998)), for example, can be used to identify potential PCR primers. Exemplary primers include primers that are 18 to 50 bases long, where at least between 18 to 25 bases are identical or complementary to at least 18 to 25 bases segment of the template sequence. Preferred template sequences for such primers are selected from a fragment of any one of SEQ ID NO: 138061 through SEQ ID NO: 195836 or complements thereof.

This invention also contemplates and provides primer pairs for amplification of nucleic acid molecules representing the ATUs. As used herein “primer pair” means a set of two oligonucleotide primers based on two separated sequence segments of a target nucleic acid sequence. One primer of the pair is a “forward primer” or “5′ primer” having a sequence which is identical to the more 5′ of the separated sequence segments. The other primer of the pair is a “reverse primer” or “3′primer” having a sequence which is complementary to the more 3′ of the separated sequence segments. A primer pair allows for amplification of the nucleic acid sequence between and including the separated sequence segments. Optionally, each primer pair can comprise additional sequences, e.g. universal primer sequences or restriction endonuclease sites, at the 5′ end of each primer, e.g. to facilitate cloning, DNA sequencing, or reamplification of the target nucleic acid sequence.

Nucleic acid molecules of the present invention include those having a nucleic acid sequence selected from the group consisting of SEQ ID NO: 138061 through SEQ ID NO: 195836 and complements thereof and fragments of either. Preferred nucleic acid molecules include those having a nucleic acid sequence selected from the following groups: SEQ ID NO: 138061 through SEQ ID NO: 162749 or complements thereof for which the principal evidence is identity to EST sequences as determined by GAP2 analysis (as indicated in more detail in Example 4 below); SEQ ID NO: 162750 through SEQ ID NO: 174652 or complements thereof for which the principal evidence is correspondence to a known peptide as determined by NAP-TBLASTX analysis (as indicated in more detail in Example 4 below); and SEQ ID NO: 174653 through SEQ ID NO: 195836 or complements thereof for which the principal evidence is a GenScan prediction (as indicated in more detail in Example 4 below). Other nucleic acid molecules of this invention are genomic nucleic acid molecules having nucleic acid sequence from within a sequence selected from the group consisting of SEQ ID NO: 1 through SEQ ID NO: 81306 or complements thereof. Still other nucleic acid molecules of this invention are EST nucleic acid molecules having a sequence selected from the group consisting of SEQ ID NO: 81307 through SEQ ID NO: 138060 or complements thereof which were produced by assembling public and original EST sequences as explained in more detail in Example 3 below. Other preferred nucleic acid molecules include any of the above groups but where such groups also include fragments of such sequences.

Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure along a sufficient portion of the molecule to allow for stable binding under laboratory hybridizing conditions. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haymes et al., Nucleic Acid Hybridization, A Practical Approach, IRL Press, Washington, D.C. (1985), the entirety of both of which are herein incorporated by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Preferred embodiments of the nucleic acid of this invention will specifically hybridize to one or more of the ATUs of this invention or complements thereof under low stringency conditions, for example at about 2.0×SSC and about 50° C. In a particularly preferred embodiment, a nucleic acid of the present invention will include those nucleic acid molecules that specifically hybridize to one or more of the ATUs of this invention or complements thereof under moderate stringency conditions. In an especially preferred embodiment, a nucleic acid of the present invention will include those nucleic acid molecules that specifically hybridize to one or more of the ATUs of this invention or complements thereof under high stringency conditions.

In another aspect of the present invention, one or more of the nucleic acid molecules of the present invention share between 100% and 90% sequence identity with one or more of the ATUs of this invention or complements thereof. In a further aspect of the present invention, one or more of the nucleic acid molecules of the present invention share between 100% and 95% sequence identity with one or more of the ATUs of this invention or complements thereof. In a more preferred aspect of the present invention, one or more of the nucleic acid molecules of the present invention share between 100% and 98% sequence identity with one or more of the ATUs of this invention or complements thereof. In an even more preferred aspect of the present invention, one or more of the nucleic acid molecules of the present invention share between 100% and 99% sequence identity with one or more of the ATUs of this invention or complements thereof.

The present invention also encompasses the use of nucleic acids of the present invention in recombinant constructs. Using methods known to those of ordinary skill in the art, an ATU sequence and/or a promoter sequence of the invention can be inserted into constructs which can be introduced into a host cell of choice for expression of the encoded protein if an ATU is used or for use of an Arabidopsis thaliana promoter to direct expression of a heterologous protein. Potential host cells include both prokaryotic and eukaryotic cells. A host cell may be unicellular or found in a multicellar differentiated or undifferentiated organism depending upon the intended use. It is understood that useful exogenous genetic material may be introduced into any non-fungal cell or organism such as a plant cell, plant, mammalian cell, mammal, fish cell, fish, bird cell, bird or bacterial cell.

Depending upon the host, the regulatory regions for expression of ATU sequences will vary, including regions from viral, plasmid or chromosomal genes, or the like. For expression in prokaryotic or eukaryotic microorganisms, particularly unicellular hosts, a wide variety of constitutive or regulatable promoters may be employed. Among transcriptional initiation regions which have been described are regions from bacterial and yeast hosts, such as E. coli, B. subtilis, Sacchromyces cerevisiae, including genes such as beta-galactosidase, T7 polymerase and tryptophan E.

Furthermore, for use in transformation of Arabidopsis thaliana, constructs may include those in which an ATU sequence or portion thereof of the present invention is positioned with respect to a promoter sequence such that production of antisense mRNA complementary to native mRNA molecules is provided. In this manner, expression of the native gene may be decreased. Such methods may find use for modification of particular functions of the targeted host, and/or for discovering the function of a protein naturally expressed in Arabidopsis thaliana.

Complements and Homologs of ATUs

Another embodiment of the present invention comprises a nucleic acid molecule which is a homolog of an ATU of this invention which encodes a polypeptide also found in a plant, animal or bacterial organism. Yet another embodiment comprises a nucleic acid molecule which encodes a polypeptide which is homologous to a polypeptide encoded by an ATU of this invention where the percent identity between the polypeptides is between about 25% and about 40%, more preferably of between about 40 and about 70%, even more preferably of between about 70% and about 90%, and even more preferably between about 90% and 99% and most preferably 100%.

Genomic sequences can be screened for the presence of protein homologs utilizing one or a number of different search algorithms that have been developed, one example of which are the suite of programs referred to as BLAST programs. In addition, unidentified reading frames may be screened for by gene prediction software such as GenScan available for downloading from the Stanford University web site. The degeneracy of the genetic code allows different nucleic acid sequences to code for the same protein or peptide, e.g. see U.S. Pat. No. 4,757,006, the entirety of which is herein incorporated by reference. As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate from the ATUs of this invention.

A further aspect of the present invention comprises one or more nucleic acid molecules which differ in nucleic acid sequence from those of an ATU of this invention due to the degeneracy in the genetic code in that they encode the same protein but differ in nucleic acid sequence or a protein having one or more conservative amino acid residue. Codons capable of coding for such conservative substitutions are known in the art. For instance, serine is a conservative substitute of alanine and threonine is a conservative substitute for serine.

Regulatory Elements

One class of agents of the present invention includes nucleic acid molecules having promoter regions or partial promoter regions or other regulatory elements, particularly those found in SEQ ID NO: 1 through SEQ ID NO: 81306 and located upstream of the trinucleotide ATG sequence at the start site of a protein coding region. As used herein, a promoter region is a region of a nucleic acid molecule that is capable, when located in cis to a nucleic acid sequence that encodes for a protein or peptide to function in a way that directs expression of one or more mRNA molecules that encodes for the protein or peptide. Promoters of the present invention can comprise nucleic acids in the range from about 300 bp to at least 1000 bp or more, say about 2000 bp or even higher say about 5000 bp and up to about 10 kb upstream of the trinucleotide ATG sequence at the start site of a protein coding region. While in many circumstances a 300 bp promoter may be sufficient for expression, additional sequences may act to further regulate expression, for example, in response to biochemical, developmental or environmental signals. In a preferred embodiment of the present invention, the promoter is upstream of a nucleic acid sequence that encodes an Arabidopsis thaliana protein homolog or fragment thereof or preferably upstream of an ATU of this invention. It is also preferred that the promoters of the present invention contain a CAAT and a TATA cis element. Moreover, the promoters of the present invention can include one or more cis elements in addition to a CAAT and a TATA box. For the most part, the promoters of the present invention will be located in contig sequences which generally represent longer nucleic acids than do singleton sequences of the present invention. Contigs in SEQ ID NO: 1 through SEQ ID NO: 81306 are recognized as those sequences whose designations begin with ATL8C, as opposed to singletons whose designations begin with ATL8S. Where an ATU is specified as being located on two different contigs, the promoter region will be located on the contig representing the 5′ region of the gene encoding sequence.

By “regulatory element” it is intended a series of nucleotides that determines if, when, and at what level a particular gene is expressed. The regulatory DNA sequences specifically interact with regulatory or other proteins. Many regulatory elements act in cis (“cis elements”) and are believed to affect DNA topology, producing local conformations that selectively allow or restrict access of RNA polymerase to the DNA template or that facilitate selective opening of the double helix at the site of transcriptional initiation. Cis elements occur within, but are not limited to promoters, and promoter modulating sequences (inducible elements). Cis elements can be identified using known cis elements as a target sequence or target motif in the BLAST programs of the present invention. Promoters of the present invention include homologs of cis elements known to effect gene regulation that show homology with the nucleic acid molecules of the present invention.

Polypeptides

Other aspects of this invention comprises one or more of the polypeptides, including proteins or peptide molecules, encoded by the coding region of an ATU of this invention or fragments thereof or homologs thereof. Protein and peptide molecules can be identified using known protein or peptide molecules as a target sequence or target motif in the BLAST programs of the present invention. In a preferred embodiment the protein or fragment molecules of the present invention are derived from Arabidopsis thaliana.

As used herein, the term “protein molecule” or “peptide molecule” includes any molecule that comprises five or more amino acids. It is well known in the art that proteins or peptides may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, ornithine, homocysteine, and homoserine.

One or more of the protein or peptide molecules may be produced via chemical synthesis, or more preferably, by expression in a suitable bacterial or eukaryotic host. Suitable methods for expression are described by Sambrook et al., Molecular Cloning, A Laboratory Manual, 2nd Edition, Cold Spring Harbor Press, Cold Spring Harbor, N.Y. (1989), or similar texts.

A “protein fragment” comprises a subset of the amino acid sequence of that protein. A protein fragment which comprises one or more additional peptide regions not derived from a base protein is a “fusion” protein. Such molecules may be derivatized to contain carbohydrate or other groups (such as keyhole limpet hemocyanin, etc.). Fusion protein or peptide molecules of the present invention are preferably produced via recombinant means.

Another class of agents comprises protein or peptide molecules encoded by the coding region of an ATU of this invention or complements thereof or, fragments or fusions thereof in which conservative, non-essential, or not relevant, amino acid residues have been added, replaced, or deleted. An example of such a homolog is the homolog protein of a non-Arabidopsis thaliana filamentous fungus. Such a homolog can be obtained by any of a variety of methods. For example, as indicated above, one or more of the disclosed sequences for primers of this invention can be used to define a pair of primers that may be used to isolate the homolog-encoding nucleic acid molecules from any desired species. Such molecules can be expressed to yield homologs by recombinant means.

Antibodies

One aspect of the present invention concerns antibodies, single-chain antigen binding molecules, or other proteins that specifically bind to one or more of the protein or peptide molecules of the present invention and their homologs, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules. In a preferred embodiment the antibodies of the present invention bind to proteins of the present invention, in a more preferred embodiment of the antibodies of the present invention bind to proteins derived from Arabidopsis thaliana.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal. It is understood that practitioners are familiar with the standard resource materials which describe specific conditions and procedures for the construction, manipulation and isolation of antibodies (see, for example, Harlow and Lane, Antibodies: A Laboratory Manual, Cold Spring Harbor Press, Cold Spring Harbor, N.Y. (1988), the entirety of which is herein incorporated by reference).

It is understood that any of the antibodies of the present invention can be substantially purified and/or be biologically active and/or recombinant.

Plant Constructs and Plant Transformants

ATUs or other nucleic acid molecules of this invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. Such genetic material may be transferred into either monocotyledons and dicotyledons including but not limited to the plants, alfalfa, Arabidopsis thaliana, barley, broccoli, cabbage, citrus, cotton, garlic, oat, oilseed rape, onion, canola, flax, maize, an ornamental plant, pea, peanut, pepper, potato, rice, rye, sorghum, soybean, strawberry, sugarcane, sugarbeet, tomato, wheat, poplar, pine, fir, eucalyptus, apple, lettuce, lentils, grape, banana, tea, turf grasses, sunflower, oil palm, etc.

Exogenous genetic material may be transferred into a plant cell by the use of a DNA vector or construct designed for such a purpose. Vectors have been engineered for transformation of large DNA inserts into plant genomes. Binary bacterial artificial chromosomes have been designed to replicate in both E. coli and Agrobacterium tumefaciens and have all of the features required for transferring large inserts of DNA into plant chromosomes. BAC vectors, e.g. a pBACwich, have been developed to achieve site-directed integration of DNA into a genome.

A construct or vector may also include a plant promoter to express the protein or protein fragment of choice. A number of promoters which are active in plant cells have been described in the literature. These include the nopaline synthase (NOS) promoter, the octopine synthase (OCS) promoter, a caulimovirus promoter such as the CaMV 19S promoter and the CaMV 35S promoter, the figwort mosaic virus 35S promoter, the light-inducible promoter from the small subunit of ribulose-1,5-bis-phosphate carboxylase (ssRUBISCO), the Adh promoter, the sucrose synthase promoter, the R gene complex promoter, and the chlorophyll a/b binding protein gene promoter. For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase (FBPase) promoter from wheat, the nuclear photosynthetic ST-LS1 promoter from potato, the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine, the promoter for the Cab-1 gene from wheat, the promoter for the CAB-1 gene from spinach, the promoter for the cab1R gene from rice, the pyruvate, orthophosphate dikinase (PPDK) promoter from Zea mays, the promoter for the tobacco Lhcb1*2 gene, the Arabidopsis thaliana SUC2 sucrose-H⁺ symporter promoter, and the promoter for the thylacoid membrane proteins from spinach (psaD, psaF, psaE, PC, FNR, atpC, atpD, cab, rbcS). Other promoters for the chlorophyll a/b-binding proteins may also be utilized in the present invention, such as the promoters for LhcB gene and PsbP gene from white mustard (Sinapis alba). Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435 and 4,633,436, all of which are herein incorporated in their entirety.

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence or the like. It is understood that one or more sequences of the present invention that act to terminate transcription may be used.

A vector or construct may also include other regulatory elements or selectable markers. Selectable markers may also be used to select for plants or plant cells that contain the exogenous genetic material. Examples of such include, but are not limited to, a neo gene which codes for kanamycin resistance and can be selected for using kanamycin, G418, etc.; a bar gene which codes for bialaphos resistance; a mutant EPSP synthase gene which encodes glyphosate resistance; a nitrilase gene which confers resistance to bromoxynil, a mutant acetolactate synthase gene (ALS) which confers imidazolinone or sulphonylurea resistance; and a methotrexate resistant DHFR gene.

A vector or construct may also include a screenable marker to monitor expression. Exemplary screenable markers include a β-glucuronidase or uidA gene (GUS), an R-locus gene, which encodes a product that regulates the production of anthocyanin pigments (red color) in plant tissues; a β-lactamase gene, a gene which encodes an enzyme for which various chromogenic substrates are known (e.g., PADAC, a chromogenic cephalosporin); a luciferase gene, a xylE gene which encodes a catechol dioxygenase that can convert chromogenic catechols; an α-amylase gene, a tyrosinase gene which encodes an enzyme capable of oxidizing tyrosine to DOPA and dopaquinone which in turn condenses to melanin; an α-galactosidase, which will turn a chromogenic α-galactose substrate. Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

Technology for introduction of DNA into cells is well known to those of skill in the art. Four general methods for delivering a gene into cells have been described: (1) chemical methods, (2) physical methods such as microinjection and bombardment, (3) viral vectors and (4) receptor-mediated mechanisms.

It is also to be understood that two different transgenic plants can also be mated to produce offspring that contain two independently segregating added, exogenous genes.

The present invention also provides for parts of the plants of the present invention. Plant parts, without limitation, include seed, endosperm, ovule and pollen. In a particularly preferred embodiment of the present invention, the plant part is a seed.

Transformation of plant protoplasts can be achieved using methods based on calcium phosphate precipitation, polyethylene glycol treatment, electroporation, and combinations of these treatments.

Any of the nucleic acid molecules of the present invention may be introduced into a plant cell in a permanent or transient manner in combination with other genetic elements such as vectors, promoters enhancers etc. Further any of the nucleic acid molecules encoding an Arabidopsis thaliana protein or fragment thereof or homologs of the present invention may be introduced into a plant cell in a manner that allows for over expression of the protein or fragment thereof encoded by the nucleic acid molecule.

Uses of the Agents of the Present Invention

Nucleic acid molecules of the present invention may be employed to obtain other Arabidopsis thaliana nucleic acid molecules. Such molecules can be readily obtained by using the above-described nucleic acid molecules to screen Arabidopsis thaliana libraries.

Nucleic acid molecules and fragments thereof of the present invention may also be employed to obtain nucleic acid molecule homologs of non-Arabidopsis thaliana species including the nucleic acid molecules that encode, in whole or in part, protein homologs of other species or other organisms, sequences of genetic elements such as promoters and transcriptional regulatory elements. Such molecules can be readily obtained by using the above-described nucleic acid molecules to screen cDNA or genomic libraries of non-Arabidopsis thaliana species. Methods for forming such libraries are well known in the art. Such homolog molecules may differ in their nucleotide sequences from those found in one or more of the Arabidopsis thaliana genes of this invention or complements thereof because complete complementarity is not needed for stable hybridization. The nucleic acid molecules of the present invention therefore also include molecules that, although capable of specifically hybridizing with the nucleic acid molecules may lack “complete complementarity.”

The disclosed nucleic acid molecules may be used to define one or more primer pairs that can be used with the polymerase chain reaction to amplify and obtain any desired nucleic acid molecule or fragment thereof. Such molecules will find particular use in generation of nucleic acid arrays, including microarrays, containing portions of or the entire encoding region for the identified Arabidopsis thaliana genes. It is noted that the molecules on such arrays may contain native intervening sequences (introns) of the genes and will still find use in microarray based methods such as transcriptional profiling for functional analysis of Arabidopsis thaliana genes and metabolic pathways.

The nucleic acid molecules of the present invention may be used for physical mapping. Physical mapping, in conjunction with linkage analysis, can enable the isolation of genes. Physical mapping has been reported to identify the markers closest in terms of genetic recombination to a gene target for cloning. Once a DNA marker is linked to a gene of interest, the chromosome walking technique can be used to find the genes via overlapping clones. For chromosome walking, random molecular markers or established molecular linkage maps are used to conduct a search to localize the gene adjacent to one or more markers. A chromosome walk is then initiated from the closest linked marker. Starting from the selected clones, labeled probes specific for the ends of the insert DNA are synthesized and used as probes in hybridizations against a representative library. Clones hybridizing with one of the probes are picked and serve as templates for the synthesis of new probes; by subsequent analysis, contigs are produced.

The degree of overlap of the hybridizing clones used to produce a contig can be determined by comparative restriction analysis. Comparative restriction analysis can be carried out in different ways all of which exploit the same principle; two clones of a library are very likely to overlap if they contain a limited number of restriction sites for one or more restriction endonucleases located at the same distance from each other. The most frequently used procedures are, fingerprinting, restriction fragment mapping or the “landmarking” technique. It is understood that the nucleic acid molecules of the present invention may in one embodiment be used in physical mapping. In a preferred embodiment, nucleic acid molecules of the present invention may in one embodiment be used in the physical mapping of Arabidopsis thaliana.

Nucleic acid molecules of the present invention can be used in comparative mapping. Comparative mapping within families provides a method to assess the degree of sequence conservation, gene order, ploidy of species, ancestral relationships and the rates at which individual genomes are evolving. Comparative mapping has been carried out by cross-hybridizing molecular markers across species within a given family. As in genetic mapping, molecular markers are needed but instead of direct hybridization to mapping filters, the markers are used to select large insert clones from a total genomic DNA library of a related species. The selected clones, each a representative of a single marker, can then be used to physically map the region in the target species. The advantage of this method for comparative mapping is that no mapping population or linkage map of the target species is needed and the clones may also be used in other closely related species. By comparing the results obtained by genetic mapping in model organisms, with those from other species, similarities of genomic structure among species can be established. Cross-hybridization of RFLP markers has been reported and conserved gene order has been established in many studies. Such macroscopic synteny is utilized for the estimation of correspondence of loci among these organisms. It is understood that nuclear acid molecules of the present invention may in another embodiment be used in comparative mapping. In a preferred embodiment the nucleic acid molecules of present invention may be used in the comparative mapping of filamentous fungi.

In one aspect of the present invention, one or more of the nucleic acid molecules of the present invention are used to determine the level (i.e., the concentration of mRNA in a sample, etc.) or pattern (i.e., the kinetics of expression, rate of decomposition, stability profile, etc.) of the expression of a protein encoded in part or whole by one or more of the nucleic acid molecule of the present invention (collectively, the “Expression Response” of a cell or tissue). As used herein, the Expression Response manifested by a cell or tissue is said to be “altered” if it differs from the Expression Response of cells or tissues of organisms not exhibiting the phenotype. To determine whether a Expression Response is altered, the Expression Response manifested by the cell or tissue of the organism exhibiting the phenotype is compared with that of a similar cell or tissue sample of a organism not exhibiting the phenotype. As will be appreciated, it is not necessary to redetermine the Expression Response of the cell or tissue sample of organisms not exhibiting the phenotype each time such a comparison is made; rather, the Expression Response of a particular organism may be compared with previously obtained values of normal organism. As used herein, the phenotype of the organism is any of one or more characteristics of an organism.

Nucleic acid molecules of the present invention can be used to monitor expression. A microarray-based method for high-throughput monitoring of gene expression may be utilized to measure gene-specific hybridization targets. This ‘chip’-based approach involves using microarrays of nucleic acid molecules as gene-specific hybridization targets to quantitatively measure expression of the corresponding genes. Every nucleotide in a large sequence can be queried at the same time. Hybridization can be used to efficiently analyze nucleotide sequences.

Several methods have been described for fabricating microarrays of nucleic acid molecules and using such microarrays in detecting nucleic acid sequences. For instance, microarrays can be fabricated by spotting nucleic acid molecules, e.g. genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Useful substrates for arrays include nylon, glass and silicon. See, for instance, U.S. Pat. Nos. 5,202,231; 5,445,934; 5,525,464; 5,700,637; 5,744,305; 5,800,992, the entirety of the disclosures of all of which are incorporated herein by reference. Sequences can be efficiently analyzed by hybridization to a large set of oligonucleotides or cDNA molecules representing a large portion of a the genes of a genome. An array consisting of oligonucleotides or cDNA molecules complementary to subsequences of a target sequence can be used to determine the identity of a target sequence, measure its amount, and detect differences between the target and a reference sequence. Nucleic acid molecule microarrays may also be screened with molecules or fragments thereof to determine nucleic acid molecules that specifically bind molecules or fragments thereof.

The microarray approach may also be used with polypeptide targets (U.S. Pat. No. 5,445,934; U.S. Pat. No. 5,143,854; U.S. Pat. No. 5,079,600; U.S. Pat. No. 4,923,901, all of which are herein incorporated by reference in their entirety). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides.

It is understood that one or more of the molecules of the present invention, preferably one or more of the nucleic acid molecules or protein molecules or fragments thereof of the present invention may be utilized in a microarray based method. In a preferred embodiment of the present invention, one or more of the Arabidopsis thaliana nucleic acid molecules or protein molecules or fragments thereof of the present invention may be utilized in a microarray based method. A particular preferred microarray embodiment of the present invention is a microarray comprising nucleic acid molecules encoding genes or fragments thereof that are homologs of known genes or nucleic acid molecules that comprise genes or fragments thereof that elicit only limited or no matches to known genes. A further preferred microarray embodiment of the present invention is a microarray comprising nucleic acid molecules having genes or fragments thereof that are homologs of known genes and nucleic acid molecules that comprise genes or fragment thereof that elicit only limited or no matches to known genes.

In a preferred embodiment, the microarray of the present invention comprises at least 1,000 or more, e.g. at least 2,000, more preferably at least 5,000 or more, e.g. at least 10,000 distinct nucleic acid molecules that specifically hybridize under high stringency to nucleic acid molecules encoding Arabidopsis thaliana protein or fragments. In a more preferred embodiment, the microarray of the present invention comprises at least 15,000, or more, e.g. at least 20,000, even more preferably at least 30,000 or more, e.g. at least about 40,000, nucleic acid molecules that specifically hybridize under high stringency to nucleic acid molecules that encode an Arabidopsis thaliana protein or fragment thereof. While it is understood that a single nucleic acid molecule may encode more than one protein or fragment thereof, in a preferred embodiment, at least 50%, preferably at least 70%, more preferably at least 80%, even more preferably at least 90% of the nucleic acid molecules that comprise the microarray encode one protein homolog or fragment thereof. It is, of course, understood that these nucleic acid molecules can be non-identical.

In a preferred embodiment, the microarray of the present invention comprises at least 1,000 or more, e.g. at least 2,000, more preferably at least 5,000 or more, e.g. at least about 10,000 distinct nucleic acid molecules that specifically hybridize under high stringency to ATUs selected from the group having SEQ ID NO: 138061 through SEQ ID NO: 195836 or fragment thereof or complement of either. In a more preferred embodiment, the microarray of the present invention comprises at least 15,000 or more, e.g. at least about 20,000, more preferably at least about 30,000 or more, e.g. at least about 40,000 distinct nucleic acid molecules that specifically hybridize under high stringency to ATUs selected from the group having SEQ ID NO: 138061 through SEQ ID NO: 195836 or fragment thereof or complement of either. While it is understood that a single nucleic acid molecule may encode more than one protein homolog or fragment thereof, in a preferred embodiment, at least 50%, preferably at least 70%, more preferably at least 80%, even more preferably at least 90% of the nucleic acid molecules that comprise the microarray encode one protein or fragment thereof.

Nucleic acid molecules of the present invention may be used in site directed mutagenesis. Site-directed mutagenesis may be utilized to modify nucleic acid sequences, particularly as it is a technique that allows one or more of the amino acids encoded by a nucleic acid molecule to be altered (e.g. a threonine to be replaced by a methionine). Three basic methods for site-directed mutagenesis are often employed, i.e. (a) cassette mutagenesis, (b) primer extension and (c) methods based on PCR. See also U.S. Pat. No. 5,880,275, U.S. Pat. No. 5,380,831, and U.S. Pat. No. 5,625,136, the entirety of all of which is incorporated herein by reference.

Any of the nucleic acid molecules of the present invention may either be modified by site-directed mutagenesis or used as, for example, nucleic acid molecules that are used to target other nucleic acid molecules for modification. It is understood that mutants with more than one altered nucleotide can be constructed using techniques that practitioners skilled in the art are familiar with such as isolating restriction fragments and ligating such fragments into an expression vector.

Preferred aspects of this invention comprise collections of genes, nucleic acid molecules, polypeptides and/or primers of this invention ranging in size from about 10 non-identical members or more, e.g. at least about 100 or 270 or higher, more preferably at least about 300 or 350, most preferably at least 500 or higher, up to about 1000, or 2000 or even higher, say about 5000, or more non-identical members. As used herein a nonidentical member is a member that differs in nucleic acid or amino acid sequence. For example, a non-identical nucleic acid molecule is a nucleic acid molecule that differs in nucleic acid sequence from the nucleic acid molecule to which it is being compared to. For example a nucleic acid molecule having the sequence 5′ CCC 3′ is not identical—i.e. non-identical—to a nucleic acid molecule having the sequence 5′ CCG 3′. In one aspect a collection may comprise all of the genes, nucleic acid molecules, polypeptides and/or primers of this invention. Such collections can be located or organized in a variety of forms, e.g. on microarrays, in solutions, in bacterial clone libraries, etc. As used herein, an “organized” collection is a collection where the nucleic acid or amino acid sequence of a member of such a collection can be determined based on its physical location.

Preferred collections of this invention comprise nucleic acid molecules having a sequence selected from SEQ ID NO: 138061 through SEQ ID NO: 195836 or homologs or complements thereof or fragments thereof, e.g. oligonucleotides including primers and primer pairs of this invention. Other embodiments of the collections of this invention comprise nucleic acid sequences, complements, homologs, fragments, oligonucleotides and primers having a sequence identified by SEQ ID NO: 138062 through SEQ ID NO. 162749, or SEQ ID NO. 162750 through SEQ ID NO: 174652 or SEQ ID NO: 174653 through SEQ ID NO: 195836 or complements thereof. Other preferred nucleic acid collections include any of the above groups but where such groups also include fragments of such sequences.

It is understood that all these preferred collections may also range in size from about 1000 or 2000 or more, e.g. at least about 3000 or 5000 non-identical nucleic acid sequences. In some embodiments the collections will comprise at least 10,000 or 15,000 or more, e.g. about 20,000 or 25,000 non-identical nucleic acid sequence. In still other embodiments the collections of this invention will comprise at least 30,000 or 40,000 or more, e.g. at least 50,000, non-identical nucleic acid sequences.

Another aspect of this invention provides the genes, nucleic acid molecules, polypeptides and/or primers in a substantially pure form. For instance, by use of the primers of this invention, any of the ATUs can be produced in substantially pure form by PCR.

Another aspect of this invention is to provide methods for determining gene expression, e.g. identifying homologous genes expressed by non-Arabidopsis thaliana organism. Such methods comprise collecting mRNA from tissue of such organism, using the mRNA as a template for producing a quantity of labeled nucleic acid, and contacting the labeled nucleic acid molecule with a collection of purified nucleic acid molecules, e.g. on a microarray.

Computer Media

One or more of the nucleotide sequence provided in SEQ ID NO: 1, through SEQ ID NO: 195836 or complements or fragments of either can be “provided” in a variety of media to facilitate use. Such a medium can also provide a subset thereof in a form that allows a skilled artisan to examine the sequences. In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; optical scanner readable medium such as printed paper; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. In addition, a variety of data processor programs and formats can be used to store the nucleotide sequence information of the present invention on computer readable medium. The sequence information can be represented in a word processing text file, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST and/or BLAZE search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 30 to 300 nucleotide residues or from about 10 to 100 of the corresponding amino acids. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequences the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

Computer media of the nucleic acid sequences of this invention can comprise a few as 1000 distinct nucleic acid sequences including complements and homologs, preferably at least 2,000 or 3,000, more preferably at least 5,000 or 10,000 or more, e.g. 15,000 or 20,000 and in certain embodiments as much as 30,00 or 40,000 distinct nucleic acid sequences.

Having now generally described the invention, the same will be more readily understood through reference to the following examples which are provided by way of illustration, and are not intended to be limiting of the present invention, unless specified.

### Example 1

### Genomic DNA Library

DNA Preparation

DNA from Arabidopsis thaliana, Landsberg erecta seedlings is prepared by a CTAB genomic DNA isolation protocol as described by Dean et al. Plant J 2:69-81 (1992) and modified by Dubois et al. Plant J. 13:141-151 (1998).

A solution of DNA to be sheared is prepared in a 1.5 ml microcentrifuge tube by mixing 15 ug of DNA, 6 μl of 10× mung bean (MB) buffer (10×MB buffer=300 mM NaOAc, pH 5.0, 500 mM NaCl, 10 mM ZnCl₂, 50% glycerol), and water to a final volume of 60 μl. The DNA solution is kept on ice prior to sonication. For sonication, a cup horn probe chilled with ice water for 1 hour prior to sonication is used. The sonicator (Ultrasonic Liquid Processor XL2020, Misonix Inc.) is pulsed for approximately 10 seconds on full power prior to use. DNA samples are sonicated twice for 6 seconds each at 60% power. Four sample tubes may be processed at once in a multi-tube rack which is positioned 1 to 3 mm above the opening in the probe. The DNA is returned to ice and a 1 μl sample is analyzed by electrophoresis on a 0.8% agarose gel in 0.5×TBE gel, run at 60 volts for 30 minutes. Sonication may be repeated if necessary.

A 0.26 μl aliquot of mung bean nuclease (150,000 u/ml) is added to sheared DNA and the sample is incubated at 30° C. for 10 minutes. To stop the digestion, 20 μl of 1 M NaCl, 140 ul dd H20, and 200 ml of phenol:chloroform are added to the sample which is then, vortexed and centrifuged for 20 minutes at 13,000 rpm. The resulting aqueous phase is transferred into a new 1.5 ml microcentrifuge tube, 500 μl of 95% ethanol is added, and the DNA is precipitated overnight at −80° C. The sample is centrifuged for 30 minutes at 13,000 rpm, washed with 500 μl of 95% ethanol and centrifuged again for 30 minutes at 13,000 rpm. The sample is then dried under vacuum, and resuspended in 10 μl TE.

The sheared DNA fragments are sized and purified by preparative agarose gel electrophoresis. Five microliters of 6×BP-XC-glycerol dye (0.25% BP, 0.25% XC, 30% glycerol) is added to the sample. The sample is split into two samples and loaded (12.5 μl per lane) on a 0.8% (1×TAE) low-melting agarose gel (SeaPlaque GTG) and electrophoresed at 60 V, 46 mA for 3.5 hours.

The gel is photographed under long wave UV and slices containing DNA fragments of 1.3-1.7 kb and 2-4 kb are excised and excess agarose cut away. The gel slices are placed in 1.5 ml microcentrifuge tubes. One gel slice is stored at −20° C. 15 μl of 1 M NaCl is added to the other gel slice, followed by melting of the agarose by incubation at 65° C. for 8 minutes. The resulting approximately 250 μl samples are placed into microcentrifuge tubes. An equal volume of water is added, following which the sample is vortexed and placed at room temperature for 2 minutes to bring the temperature up to 30-35° C. 0.5 ml of water-saturated phenol that has been cooled on ice is added and the sample vortexed vigorously. The sample is placed on ice for 5 minutes, and the vortexing step repeated.

The sample is centrifuged at 4° C. in a microcentrifuge for 20 minutes. The upper phase is transferred to a clean tube, and the bottom phenol layer is reextracted by addition of 200 μl of dd H2O. The sample is vortexed and placed on ice for 5 minutes, followed by centrifugation for 15 minutes. The aqueous layer is extracted and added to the aqueous layer from the previous step. Phenol extraction is repeated with 0.5 ml phenol, followed by vortexing and centrifugation for 20 minutes at 4° C. The aqueous layer is removed and repeated sec-butanol extractions are performed until the final volume is reduced to approximately 0.165 ml

Two volumes of 95% ethanol (400 μl) are added and the sample is stored at −80° C. overnight. The sample is centrifuged for 30 minutes at room temperature to pellet the DNA, washed once with 95% ethanol and dried briefly under vacuum. The sample is resuspended in 7 μl of TE. A 1 ul sample is run on a 0.8% agarose gel with markers to estimate concentration of recovered fraction.

M13 Library

20 ng of M13 DNA digested with SmaI is mixed with 1 μl of 10× ligation buffer (10× ligation buffer=0.5M tris pH 7.4, 0.1M MgCl₂, 0.1M DDT), 1 μl of 1 mM ATP and 100-200 ng of sheared genomic DNA fragments (1-3 μl volume), and 0.3 μl of high concentration NEB ligase (5 unit/μl) is added. Water is added to a final volume of 10 ul and the sample is incubated overnight at 14° C.

Plasmid Library

200 ng (4 μl) of pSTBlue vector (Novegene) is mixed with approximately 600 ng (12 μl) of sheared genomic DNA fragments from the 2-4 kb size range gel slices and 1.2 μl of Gibco T4 ligase (5 units per μl) is added. Water is added to a final volume of 30 ul and the sample is incubated overnight at 14° C.

Transformation

The ligation reaction is titered and diluted for optimal transformation efficiency. When the ligation contains approximately 20 ng of M13 vector, the dilution will typically be from 1:25 to 1:100. A 1:25 dilution is used for plasmid ligation containing approximately 200 ng of vector DNA. To increase transformation efficiency, the ligase is denatured by heating at 65° C. for 7 minutes, and placed at room temperature for 5 minutes following the heating step.

A sterile electroporation cuvette is chilled for each transformation. Electro-competent cells are removed from the −80° C. freezer and thawed on ice. For each M13 transformation, a sterile tube containing 25 ul of IPTG (25 mg/ml in water), 25 μl of X-Gal (25 mg/ml in dimethylformamide) and 3 ml of YT top agar is prepared, capped and placed in a 45° C. water bath. YT plates are pre-warmed at 37° C. for several hours to avoid cross-contamination problems that may result if water remains on plates. For plasmid transformations, a sterile tube containing 0.5 ml of SOC medium is prepared for each transformation, and L+amp plates are pre-spread with 25 μl of IPTG and 25 μl of X-Gal.

25 μl of electro-competent cells are mixed with DNA in diluted ligation mix in the cuvette, and the sample pulsed in an E. coli pulser (BioRad) set to the appropriate voltage (1.80 kV for 0.1 cm cuvettes; 2.50 kV for 0.2 cm cuvettes). The cuvette is removed from the pulser, and the sample immediately transferred to the tube containing SOC or YT top agar. For M13 transfections, the sample is plated immediately on YT plates. For plasmid transformations, the tube is placed in a 37° C. shaker for 15-30 minutes and 30 ul aliquots are plated on L+Amp plates. Plates are incubated at 37° C. overnight.

### Example 2

Two basic methods can be used for DNA sequencing, the chain termination method of Sanger et al., Proc. Natl. Acad. Sci. (U.S.A.) 74:5463-5467 (1977), the entirety of which is herein incorporated by reference and the chemical degradation method of Maxam and Gilbert, Proc. Natl. Acad. Sci. (U.S.A.) 74:560-564 (1977), the entirety of which is herein incorporated by reference. Automation and advances in technology such as the replacement of radioisotopes with fluorescence-based sequencing have reduced the effort required to sequence DNA (Craxton, Methods 2:20-26 (1991), the entirety of which is herein incorporated by reference; Ju et al., Proc. Natl. Acad. Sci. (U.S.A.) 92:4347-4351 (1995), the entirety of which is herein incorporated by reference; Tabor and Richardson, Proc. Natl. Acad. Sci. (U.S.A.) 92:6339-6343 (1995), the entirety of which is herein incorporated by reference). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y., the entirety of which is herein incorporated by reference).

PHRED is used to call the bases from the sequence trace files (http://www.mbt.washington.edu). PHRED uses Fourier methods to examine the four base traces in the region surrounding each point in the data set in order to predict a series of evenly spaced predicted locations. That is, it determines where the peaks would be centered if there were no compressions, dropouts, or other factors shifting the peaks from their “true” locations. Next, PHRED examines each trace to find the centers of the actual, or observed peaks and the areas of these peaks relative to their neighbors. The peaks are detected independently along each of the four traces so many peaks overlap. A dynamic programming algorithm is used to match the observed peaks detected in the second step with the predicted peak locations found in the first step.

After the base calling is completed, two sequence quality steps occur 1) poor quality end sequences are cut and if the resulting sequence is 50 bp or less it is deleted 2) overall sequence quality is examined and poor sequences are deleted from the data set if they have an average quality cutoff below 12.5. Contaminating sequences (E. coli, yeast, vector, linker) are removed after sequence quality assessment. The screened file contained 498,037 sequences.

Contigs are assembled using PANGEA clustering tools (PANGEA SYSTEMS. INC) and PHRAP (http://www.mbt.washington.edu). PANGEA clustering tools are a series of scripts which group sequences (clusters) by comparing pairs of sequences for overlapping bases. The overlap is determined using the following high stringency parameters: word size=8; window size=60; and identity is 93%. Each of the clusters are then assembled using PHRAP. This step results in 102,349 islands. The next step is to combine the islands together to collapse the contig number even further. Default, less stringent parameters, are used in this step: minimum match=14, minimum score=30; and the penalty is −2. The resulting, final, total number of islands equals 81,306. Of these, 50,262 are actual contigs and 31,044 are singletons. The final set of 81,306 nucleic acid sequences is identified in Table 1 by sequence identification (Seq. ID.) “ATL8Sxxxxx” (for singleton sequences) and “ATL8Cxxxxx” (for contig sequences) and by the corresponding sequence number (SEQ ID NO: 1 through SEQ ID NO: 81,306). The final set of 81,306 genomic sequences is run through the following annotation and gene selection processes described in Example 4. The genomic sequence traces and many of the contigs and singleton traces are disclosed in copending provisional applications for patent identified by Ser. Nos. 60/111,990; 60/111,991; 60/114,151; 60/120,644; 60/135,825; 60/139,932; 60/143,994 and 60/155,422.

### Example 3

This example illustrates the generation of the ATCEA4 EST library from cDNA prepared from a variety of Arabidopsis thaliana, Columbia ecotype, and tissue. Wild type Arabidopsis thaliana seeds are planted in commonly used planting pots and grown in an environmental chamber. Tissue is harvested as follows:
(a) For leaf tissue-based cDNA, leaf blades are cut with sharp scissors at seven weeks after planting;
(b) For root tissue-based cDNA, roots of seven-week old plants are rinsed intensively with tap water to wash away dirt, and briefly blotted by paper towel to take away free water;
(c) For stem tissue-based cDNA, stems are collected seven to eight weeks after planting by cutting the stems from the base and cutting the top of the plant to remove the floral tissue;
(d) For flower bud tissue-based cDNA, green and unopened flower buds are harvested about seven weeks after planting;
(e) For open flower tissue-based cDNA, completely opened flowers with all parts of floral structure observable, but no siliques are appearing, and are harvested about seven weeks after planting;
(f) For immature seed tissue-based cDNA, seeds are harvested at approximately 7-8 weeks of age. The seeds range in maturity from the smallest seeds that could be dissected from siliques to just before starting to turn yellow in color.

All tissue is immediately frozen in liquid nitrogen and stored at −80° C. until total RNA extraction. The stored RNA is purified using Trizol reagent from Life Technologies (Gibco BRL, Life Technologies, Gaithersburg, Md. U.S.A.), essentially as recommended by the manufacturer. Poly A+ RNA (mRNA) is purified using magnetic oligo dT beads essentially as recommended by the manufacturer (Dynabeads, Dynal Corporation, Lake Success, N.Y. U.S.A.).

Construction of plant cDNA libraries is well-known in the art and a number of cloning strategies exist. A number of cDNA library construction kits are commercially available. The Superscript™ Plasmid System for cDNA synthesis and Plasmid Cloning (Gibco BRL, Life Technologies, Gaithersburg, Md. U.S.A.) is used, following the conditions suggested by the manufacturer.

The cDNA libraries are plated on LB agar containing the appropriate antibiotics for selection and incubated at 37° for a sufficient time to allow the growth of individual colonies. Single colonies are individually placed in each well of a 96-well microtiter plates containing LB liquid including the selective antibiotics. The plates are incubated overnight at approximately 37° C. with gentle shaking to promote growth of the cultures. The plasmid DNA is isolated from each clone using Qiaprep plasmid isolation kits, using the conditions recommended by the manufacturer (Qiagen Inc., Santa Clara, Calif. U.S.A.).

The template plasmid DNA clones are used for subsequent sequencing. For sequencing the cDNA libraries, a commercially available sequencing kit, such as the ABI PRISM dRhodamine Terminator Cycle Sequencing Ready Reaction Kit with AmpliTaq® DNA Polymerase, FS, is used under the conditions recommended by the manufacturer (PE Applied Biosystems, Foster City, Calif.). The ESTs of the present invention are generated by sequencing initiated from the 5′ end of each cDNA clone.

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y., the entirety of which is herein incorporated by reference).

The generated ESTs (including any full length cDNA sequences) are combined with ESTs and full length cDNA sequences in public databases such as GenBank. Duplicate sequences are removed; and, duplicate sequence identification numbers are replaced. The combined dataset is then clustered and assembled using Pangea Systems tool identified as CAT v.3.2. First, the EST sequences are screened and filtered, e.g. high frequency words are masked to prevent spurious clustering; sequence common to known contaminants such as cloning bacteria are masked; high frequency repeated sequences and simple sequences are masked; unmasked sequences of less than 100 bp are eliminated. The thus-screened and filtered ESTs are combined and subjected to a word-based clustering algorithm which calculates sequence pair distances based on word frequencies and uses a single linkage method to group like sequences into clusters of more than one sequence, as appropriate. Clustered sequence files are assembled individually using an iterative method based on PHRAP/CRAW/MAP providing one or more self-consistent consensus sequences and inconsistent singleton sequences. The assembled clustered sequence files are checked for completeness and parsed to create data representing each consensus contiguous sequence (contig), the initial EST sequences, and the relative position of each EST in a respective contig. The sequence of the 5′ most clone is identified from each contig. The initial sequences that are not included in a contig are separated out. A FASTA file is created consisting of 56,754 sequences comprising the sequence of each contig and all original sequences which were not included in a contig. The FASTA file is referred to as the ATCEA4 EST library. The EST contigs and original sequences which are not included in a contig are presented in Table 2 comprising SEQ ID No: 81,307 through SEQ ID NO: 138,060.

### Example 4

This example illustrates the identification of ATUs within the ATL8 genomic contig and singleton library assembled in Example 2. The genes and partial genes embedded in such contigs are identified through a series of informatic analyses. The tools to define genes fall into two categories: homology-based and predictive-based methods. Homology-based searches (e.g., GAP2 and BLASTX supplemented by NAP) detect conserved sequences during comparisons of DNA sequences or hypothetically translated protein sequences to public and/or proprietary DNA and protein databases. Existence of an Arabidopsis thaliana gene is inferred if significant sequence similarity extends over the majority of the target gene. Since homology-based methods may overlook genes unique to Arabidopsis thaliana, for which homologous nucleic acid molecules have not yet been identified in databases, gene prediction programs are also used. Predictive methods employed in the definition of the Arabidopsis thaliana genes included the use of the GenScan gene predictive software program which is available from Stanford University (e.g. at the web site http://gnomic/stanford.edu/GENSCANW.html). GenScan, in general terms, infers the presence and extent of a gene through a search for “gene-like” grammar.

The homology-based methods used to define the Arabidopsis thaliana gene set included GAP2 and BLASTX supplemented by NAP. For a description of BLASTX see Coulson, Trends in Biotechnology 12:76-80 (1994) and Birren et al., Genome Analysis, 1:543-559 (1997). GAP2 and NAP are part of the Analysis and Annotation Tool (AAT) for Finding Genes in Genomic Sequences which was developed by Xiaoqiu Huang at Michigan Tech University and is available at the web site http://genome.cs.mtu.edu/. The AAT package includes two sets of programs, one set DPS/NAP (referred to as “NAP”) for comparing the query sequence with a protein database, and the other set DDS/GAP2 (referred to as “GAP2”) for comparing the query sequence with a cDNA database. Each set contains a fast database search program and a rigorous alignment program. The database search program quickly identifies regions of the query sequence that are similar to a database sequence. Then the alignment program constructs an optimal alignment for each region and the database sequence. The alignment program also reports the coordinates of exons in the query sequence. See Huang, et al., Genomics 46: 37-45 (1997).

The GAP2 program computes an optimal global alignment of a genomic sequence and a cDNA sequence without penalizing terminal gaps. A long gap in the cDNA sequence is given a constant penalty. The DNA-DNA alignment by GAP2 adjusts penalties to accommodate introns. The GAP2 program makes use of splice site consensuses in alignment computation. GAP2 delivers the alignment in linear space, so long sequences can be aligned. See Huang, Computer Applications in the Biosciences 10 227-235 (1994). The GAP2 program aligned the Arabidopsis thaliana Landsberg erecta contigs with the ATCEA4 library of Arabidopsis thaliana Columbia ESTs prepared as described above in Example 3.

The NAP program computes a global alignment of a DNA sequence and a protein sequence without penalizing terminal gaps. NAP handles frameshifts and long introns in the DNA sequence. The program delivers the alignment in linear space, so long sequences can be aligned. It makes use of splice site consensuses in alignment computation. Both strands of the DNA sequence are compared with the protein sequence and one of the two alignments with the larger score is reported. See Huang, and Zhang, “Computer Applications in the Biosciences 12(6), 497-506 (1996).

NAP takes a nucleotide sequence, translates it in three forward reading frames and three reverse complement reading frames, and then compares the six translations against a protein sequence database (e.g. the non-redundant protein (i.e., nr-aa) database maintained by the National Center for Biotechnology Information as part of GenBank and available at the web site: http://www.ncbi.nlm.nih.gov).

The first homology-based search for genes in the Arabidopsis thaliana contigs is effected using the GAP2 program and the ATCEA4 library of clustered Arabidopsis thaliana Columbia ESTs. The Arabidopsis thaliana Columbia EST clusters represented by SEQ ID NO. 81,307 through SEQ ID NO. 138060 are mapped onto an assembly of Arabidopsis thaliana Landsberg contigs represented by SEQ ID NO. 1 through SEQ ID NO. 81,306 using the GAP2 program. GAP2 standards for selecting a DNA-DNA match were ≧82% sequence identity with the following parameters:

gap extension penalty=1

match score=2

gap open penalty=6

gap length for constant penalty=20

mismatch penalty=−2

minimum exon length=21

When a particular ATCEA4 EST aligns to more than one ATL8 contig, the alignment with the highest identity is selected and alignments with lower levels of identity are filtered out as surreptitious alignments. ATCEA4 EST sequences aligning to ATL8 contigs with exceptionally low complexity were filtered out when the basis for alignment included a high number of ESTs with poly A tails aligning to genomic regions with extended repeats of A or T.

The second homology-based method used for gene discovery is BLASTX hits extended with the NAP software package. BLASTX is run with the Arabidopsis thaliana genomic contigs represented by SEQ ID NO. 1 through SEQ ID NO. 81,306 as queries against the GenBank non-redundant protein data library identified as “nr-aa”. NAP is used to better align the amino acid sequences as compared to the genomic sequence. NAP extends the match in regions where BLASTX has identified high-scoring-pairs (HSPs), predicts introns, and then links the exons into a single ORF prediction. Experience suggests that NAP tends to mis-predict the first exon. The NAP parameters are:

gap extension penalty=1

gap open penalty=15

gap length for constant penalty=25

min exon length (in aa)=7

homology >30%

The NAP alignment score and GenBank reference number for best match are reported for each ATU for which there is a NAP hit.

The GenScan program is “trained” with Arabidopsis thaliana characteristics. Though better than the “off-the-shelf” version, the GenScan trained to identify Arabidopsis thaliana genes proved more proficient at predicting exons than predicting full-length genes. Predicting full-length genes is compromised by point mutations in the unfinished contigs, as well as by the short length of the contigs relative to the typical length of a gene. Due to the errors found in the full-length gene predictions by GenScan, inclusion of GenScan-predicted genes is limited to those genes and exons whose probabilities are above a conservative probability threshold. The GenScan parameters are:

weighted mean GenScan P value >0.4

mean GenScan T value >0

mean GenScan Coding score >50

length >200 bp

minimum TBLASTX E value <1E-20

The weighted mean GenScan P value is a probability for correctly predicting ORFs or partial ORFs and is defined as the (1/Σlᵢ)(ΣlᵢPᵢ), where “l” is the length of a exon and “P” is the probability or correctness for the exon. The weighted mean GenScan probability for all ATUs is reported in Table 3.

ORF indications from GAP2-EST, NAP and GenScan are assembled into candidate gene clusters by first splitting the indicated ORFs into forward (+) and reverse (−) strand indication groups for each contig. Overlapping indications or indications with less than 150 bp intervening were grouped together (150 bp is less than the expected intergenic distance for genes on the same strand). The principle evidence that a given cluster represents a particular ORF or partial ORF is based primarily on the type of indication (GAP2-EST, NAP-BLASTX and GenScan), secondarily on the score of the indication and thirdly on the consensus sequence length. The order of confidence for type of indication is highest for GAP2-EST, next for NAP-BLASTX and lowest for GenScan. Clusters where then collapsed. Using clonemate information for the ATCEA4 EST library, when more than one potential ATU aligned to ATCEA4 EST sequences which were assembled from sequences of two or more common clone ancestors, the potential ATUs were collapsed into a single ORF possibly spanning more than one ATL8 genomic contig. When two potential ATUs had indications for an ORF of the same sequence but on different strands and one of the potential ATUs was based solely on EST evidence, the EST strand indication was declared suspect as being of improper orientation and the EST was agglomerated into the other potential ATU.

The potential ATUs were then ranked and sorted, the highest score went to clustered sequence indicated by all three methods GAP2-EST, NAP-BLASTX and GenScan and the lowest score to sequence indicated solely by GenScan. In Table 3 the ATUs of this invention are identified in the sequence identification (seq. id.) column with the name ATU (Arabidopsis thaliana unigene) and begins with ATU00001 for SEQ ID NO. 138,061. The ATUs identified primarily by GAP-EST are identified by “gap2” in the “principal evidence” column of Table 3 and include SEQ ID No: 138,061 through SEQ ID No: 162,749. The ATUs identified primarily by NAP, i.e. in the absence of a GAP2-EST alignment are identified by “nap” in the principal evidence column of Table 3 and include SEQ ID NO. 162,750 through SEQ ID NO. 174,652. The ATUs identified solely by GenScan are identified by “GENSCAN” in the principal evidence column of Table 3 and include SEQ ID NO. 174,653 through SEQ ID NO. 195,836.

### Example 5

This example serves to illustrate the design of primers of this invention which are useful, for instance, for initiating synthesis of nucleic acid molecules of this invention, specifically substantial parts of certain ATUs of this invention. Such primers are designed with the program Primer3 (obtained from the MIT-Whitehead Genome Center) with a “perl-oracle” wrapper. The criteria applied to design a primer include:

Primer annealing temperature (minimum 65° C., optimum 70° C., maximum 75° C.)

Primer length (minimum 18 bp, optimum 20 bp, maximum 28 bp)

G+C content (minimum 20%, maximum 80%)

Position of the primer relative to the gene

Length of the amplified region (500 to 800 bp)

PHRED quality score of the gene template (minimum of 20)

Whether the gene was defined from one or two contigs

Maximum mismatch=12.0 (weighted score from Primer3 program)

Pair Max Misprime=24.0 (weighted score from Primer3 program)

Maximum N's=0

Maximum poly-X=5

The primary goal of the design process is the creation of groups of primer pairs with a common annealing temperature (Tₘ). When the program could identify a primer pair for any gene that fit the criteria, the gene is removed from the bin of genes needing primer design. Genes remaining in the bin are subjected to additional rounds of primer-picking, with the gradual and simultaneous relaxation of the criteria (i.e., lowering the annealing temperature, increasing the size of the window where primers could be predicted, expanding the range of permitted size and G+C content, removing the need for a G/C clamp), until a sufficient number of primers are picked for the ATUs of this invention. After the Arabidopsis thaliana specific portion of the primers is selected, an additional common primer tail sequence (universal primer) is added to the 5′ ends. For the forward primers, the additional common bases added are: (5′-GAATTCACTGCGGCCGCCATG-3′); for the reverse primers the additional common bases added are: (5′-GTTCTCGAGACGAGCGATCGC-3′). The universal primer tail sequences are added so that subsequent reamplifications of any primer pair can be done with a single set of primers. In addition, the primer tail sequences contain restriction digestion sites for 8 bp cutters (NotI and SgfI) and 6 bp cutters (EcoRI and XhoI) to facilitate cloning of ATUs into vectors. The forward primers contains EcoRI and NotI restriction sites; the reverse primers contains XhoI and SgfI restriction sites. It is noted that primer pairs are not required to contain the universal tail sequence, the relevant portion for amplification and/or hybridization probes being the Arabidopsis thaliana specific sequences.

Other modifications of the above described embodiments of the invention which are obvious to those of skill in the area of molecular biology and related disciplines are intended to be within the scope of the following claims.

LENGTHY TABLE REFERENCED HERE
US20100255584A1-20101007-T00001
Please refer to the end of the specification for access instructions.

LENGTHY TABLE REFERENCED HERE
US20100255584A1-20101007-T00002
Please refer to the end of the specification for access instructions.

LENGTHY TABLE REFERENCED HERE
US20100255584A1-20101007-T00003
Please refer to the end of the specification for access instructions.

### Table Column Heading Descriptions

Table 1

Seq No.

Provides the SEQ ID NO. for the listed sequences.

Seq ID

Arbitrary identification assigned to each contig or singleton of genomic sequuence. contigs designations begin with ATL8C. Singleton designations begin with ATL8S.

Table 2

Seq No.

Provides the SEQ ID NO. for the listed sequences.

Seq ID

Arbitrarily assigned number for each clustered EST Arabidopsis thaliana Columbia. EST contig designations begin with ATCEA4.

Table 3

Seq No.

Provides the SEQ ID NO. for the listed sequences.

Seq ID

Arbitrarily assigned number for each ATU (Arabidopsis thaliana unigene).

Unigene location

Indicates genomic contigs or singletons from which the ATUs are identified and the location of the ATU within the contig or singleton. In cases where the first numeral is higher than its corresponding second numeral, the Arabidopsis thaliana protein or fragment thereof is encoded by the complement of the sequence set forth in the sequence listing. The first numeral separated from the contig or singleton ID by a colon represents the starting point for the codon for the most N-terminal (if the first number is lower than the second number) or C-terminal (if the first number is higher than the second number) amino acid for the protein or protein fragment encoded by the ATU.

Principal evidence

A code which identifies the most reliable ATU selection method for the particular ATU. The selection methods are described in detail in Example 4 and briefly summarized as follows:
gap2: GAP2 identified ORFs
nap: NAP predicted ORFs
GENSCAN: Genscan prediction

ESTs aligned

Indicates ATCEA4 EST library entry (ies) for sequence(s) which matched to the Arabidopsis thaliana contig query.

EST pct identities

Indicates the percent identity for the EST alignment(s).

Genscan prediction

Indicates genomic contigs or singletons from which the ATUs are predicted by GenScan and the location of the ATU within the contig or singleton. In cases where the first numeral is higher than its corresponding second numeral, the Arabidopsis thaliana protein or fragment thereof is encoded by the complement of the sequence set forth in the sequence listing. The first numeral separated from the contig or singleton ID by a colon represents the starting point for the codon for the most N-terminal (if the first number is lower than the second number) of C-terminal (if the first number is higher than the second number) amino acid for the protein or protein fragment encoded by the ATU.

Genscan weighted p score

Indicates the weighted mean GenScan probability for correctness of the prediction.

Ncbi gids

Refers to National Center for Biotechnology Information GenBand Identifier number which is the best match for a given contig or singleton region from which the associated ATU was identified using NAP.

Nap identities

The percentage of identically matched nucleotides (or residues) that exist along the length of that portion of the sequence which is aligned by the BLAST comparison to generate the statistical scores presented.

Nap scores

The aat_nap score is reported by the NAP program in the AAT package. It is an alignment score in which each match and mismatch is scored based on the BLOSUM62 scoring matrix.

Blastx pscores

A score that is generated by sequence comparison of the designated clone with the designated GenBank sequence.

Genbank description

A description of the database entry referenced in the “NAP hit” column.

LENGTHY TABLE
The patent application contains a lengthy table section. A copy of the table is available in electronic form from the USPTO web site (http://seqdata.uspto.gov/?pageRequest=docDetail&DocID=US20100255584A1). An electronic copy of the table will also be available from the USPTO upon request and payment of the fee set forth in 37 CFR 1.19(b)(3).

We claim:

1. A substantially purified nucleic acid molecule comprising an Arabidopsis thaliana EST selected from the group consisting of SEQ ID NO: 81307 through SEQ ID NO: 138060 and complements thereof.

2. A substantially purified nucleic acid molecule of the Arabidopsis thaliana genome having a nucleic acid sequence selected from the group consisting of SEQ ID NO: 138601 through SEQ ID NO: 195836 and complements thereof.

3. The substantially purified nucleic acid molecule according to claim 2, wherein said group consists of SEQ ID NO: 138601 through SEQ ID NO: 162749 and complements thereof.

4. The substantially purified nucleic acid molecule according to claim 2, wherein said group consists of SEQ ID NO: 162749 through SEQ ID NO: 174652 and complements thereof.

5. The substantially purified nucleic acid molecule according to claim 2 wherein said group consists of SEQ ID NO: 174653 through SEQ ID NO: 195836 and complements thereof

6. The substantially purified nucleic acid molecule according to claim 2, wherein said nucleic acid molecule further comprises nucleic acid sequences comprising one or more of a promoter region, regulatory region or intron region or parts of said regions.

7. A substantially purified first nucleic acid molecule which is complementary to a second nucleic acid molecule of the Arabidopsis thaliana genome having a nucleic acid sequence selected from the group consisting of SEQ ID NO: 138061 through SEQ ID NO: 195835 and complements thereof wherein said first nucleic acid molecule and said second nucleic acid molecule hybridize to one another with sufficient stability to remain annealed to one another under at least low stringency conditions of washing with a salt solution having a concentration of about 2.0× sodium chloride/sodium citrate (SSC) at 50° C.

8. The substantially purified first nucleic acid molecule according to claim 7, wherein said stringency conditions are at least 0.2×SSC at 50° C.

9. The substantially purified first nucleic acid molecule according to claim 7, wherein said nucleic acid molecule of the Arabidopsis thaliana genome has a sequence selected from the group consisting of SEQ ID NO: 138061 through SEQ ID NO: 162749 and complements thereof.

10. The substantially purified first nucleic acid molecule according to claim 7, wherein said second nucleic acid molecule of the Arabidopsis thaliana genome has a sequence selected from the group consisting of SEQ ID NO: 162750 through SEQ ID NO: 174652 and complements thereof.

11. The substantially purified first nucleic acid molecule according to claim 7, wherein said second nucleic acid molecule of the Arabidopsis thaliana genome has a sequence selected from the group consisting of SEQ ID NO: 174653 through SEQ ID NO: 195836 and complements thereof.

12. A substantially purified first nucleic acid molecule which is homologous to a second nucleic acid molecule having a nucleic acid sequence selected from the group consisting of SEQ ID NO: 138061 through SEQ ID NO: 195836 and complements thereof, wherein at least 90% of the nucleic acid sequence of said substantially purified first nucleic acid molecule is identical to said second nucleic acid molecule.

13. The substantially purified first nucleic acid molecule according to claim 12, wherein said first nucleic acid sequence is 100% identical to a nucleic acid sequence of a non-Arabidopsis thaliana homologue.

14. The substantially purified first nucleic acid molecule according to claim 12, wherein at least 98% of the sequence of said substantially purified nucleic acid molecule is identical to said second nucleic acid molecule.

15. The substantially purified first nucleic acid molecule according to claim 12, wherein said second nucleic acid has a sequence selected from the group consisting of SEQ ID NO: 138601 through SEQ ID NO: 162749 and complements thereof.

16. The substantially purified first nucleic acid molecule according to claim 12, wherein said second nucleic acid has a sequence selected from the group consisting of SEQ ID NO: 162749 through SEQ ID NO: 174652 and complements thereof.

17. The substantially purified first nucleic acid molecule according to claim 12, wherein said second nucleic acid has a sequence selected from the group consisting of SEQ ID NO: 174653 through SEQ ID NO: 195836 and complements thereof

18. A substantially purified polypeptide encoded by a nucleic acid sequence selected from the group consisting of SEQ ID NO: 138061 through SEQ ID NO: 195836.

19. A transformed cell or organism cell or plant having an exogenous nucleic acid molecule which comprises:
(a) a promoter region which functions in said cell to cause the production of a mRNA molecule; which is linked to
(b) a structural nucleic acid molecule which is homologous or complementary to a nucleic acid molecule according to claim 2, which is linked to
(c) a 3′ non-translated sequence that functions in said cell to cause termination of transcription and addition of polyadenylated ribonucleotides to a 3′ end of said mRNA molecule.

20. A transformed cell or organism according to claim 19 which is selected from the group consisting of a plant cell, plant, mammalian cell, mammal, fish cell, fish, bird cell, bird, bacterial cell and fungal cell and wherein said mRNA encodes a protein in said cell.

21. A transformed cell or organism according to claim 19, wherein said structural nucleic acid molecule is a transcribed nucleic acid molecule with a transcribed strand and a non-transcribed strand and the transcribed strand specifically hybridizes to an mRNA molecule.

22. Computer readable medium having recorded thereon at least 1000 of the nucleotide sequences depicted in SEQ ID NO: 138061 through SEQ ID NO: 195836 or complements thereof.

23. Computer readable medium according to claim 22 having recorded thereon at least 10,000 said nucleotide sequences.

24. A transformed cell or organism having an exogenous nucleic acid molecule which comprises:
(a) a promoter region which functions in said cell to cause the production of an mRNA molecule wherein said promoter nucleic acid molecule is selected from the group consisting of a promoter located within SEQ ID NO: 1 through SEQ ID NO: 81306 or complements thereof upstream of a gene having a nucleic acid sequence selected from the group consisting of SEQ ID NO: 138061 through SEQ ID NO: 195836; which is linked to
(b) a structural nucleic acid molecule encoding a protein or peptide; which is linked to
(c) a 3′ non-translated nucleic acid sequence that functions in said cell to cause termination of transcription and addition of polyadenylated ribonucleotides to a 3′ end of said mRNA molecule.

25. A transformed cell or organism according to claim 24 which is selected from the group consisting of a plant cell, plant, mammalian cell, mammal, fish cell, fish, bird cell, bird, bacterial cell and fungal cell and wherein said mRNA encodes a protein in said cell.

26. A transformed cell or organism having an exogenous nucleic acid molecule which comprises a structural nucleic acid sequence which expresses an mRNA which is complementary to and hybridizes to at least part of a nucleic acid molecule having a sequence selected from the group consisting of SEQ ID NO: 138061 through SEQ ID NO: 195836 and homologs thereof.

27. A substantially purified oligonucleotide nucleic acid molecule comprising between about 15 and about 100 nucleotides homologous or complementary to a nucleotide sequence within any of SEQ ID NO: 138061 through SEQ ID NO: 195836.

28. A oligonucleotide nucleic acid molecule according to claim 27 comprising in the range of 18 to 50 bases, wherein from 18 to 25 of said bases are identical or complementary to an 18-25 bp segment of sequences from a fragment of SEQ ID NO: 138061 through SEQ ID NO: 195836.

29. A oligonucleotide nucleic acid molecule according to claim 28 wherein said 18 to 25 of said bases are identical or complementary to an 18-25 bp segment of sequences from a fragment of SEQ ID NO: 138061 through SEQ ID NO: 162749.

30. A oligonucleotide nucleic acid molecule according to claim 28 wherein said 18 to 25 of said bases are identical or complementary to an 18-25 bp segment of sequences from a fragment of SEQ ID NO: 162750 through SEQ ID NO: 174652.

31. A oligonucleotide nucleic acid molecule according to claim 28 wherein said 18 to 25 of said bases are identical or complementary to an 18-25 bp segment of sequences from a fragment of SEQ ID NO: 174653 through SEQ ID NO: 195836.

32. A collection of at least 1000 non-identical oligonucleotides according to claim 27.

33. A collection of at least 2000 non-identical oligonucleotides according to claim 27.

34. A collection of at least 5000 non-identical oligonucleotides according to claim 27.

35. A collection of at least 10,000 non-identical oligonucleotides according to claim 27.

36. A collection of at least 15,000 non-identical oligonucleotides according to claim 27.

37. A collection of at least 20,000 non-identical oligonucleotides according to claim 27.

38. A collection according to claim 32 wherein said oligonucleotides are situated in an array on a substrate.

39. A primer pair for amplification of a nucleic acid molecule of SEQ ID NO: 138061 through SEQ ID NO: 195836 comprising oligonucleotides according to claim 27.

40. A collection of purified nucleic acid molecules generated from a DNA template of the Arabidopsis thaliana genome using a collection of primer pairs according to claim 39, wherein said collection of purified nucleic acid molecules comprises at least 2 non-identical purified nucleic acid molecules.

41. A collection according to claim 40 wherein said purified nucleic acid molecules are generated by polymerase chain reaction.

42. A collection according to claim 41 comprising at least about 1000 non-identical nucleic acid molecules.

43. A collection according to claim 41 comprising at least about 2000 non-identical nucleic acid molecules.

44. A collection according to claim 41 comprising at least about 50000 non-identical nucleic acid molecules.

45. A collection according to claim 41 comprising at least about 15,000 non-identical nucleic acid molecules.

44. A collection according to claim 41 comprising at least about 20,000 non-identical nucleic acid molecules.

45. A collection according to claim 41 comprising at least about 30,000 non-identical nucleic acid molecules.

46. A collection according to claim 41 wherein said purified nucleic acid molecules are situated in an array on a substrate.

47. A collection of at least 3000 non-identical purified nucleic acid molecules having nucleic acid sequences selected from the group consisting of
(a) SEQ ID NO: 138061 through SEQ ID NO: 195836;
(b) sequences which are complementary to the nucleic acid sequences of group (a), wherein said purified nucleic acid molecules hybridize to nucleic acid molecules of the Arabidopsis thaliana genome having a sequence of a complement of group (a) with sufficient stability to remain annealed to one another under at least low stringency conditions of washing with a salt solution having a concentration of about 0.2 sodium chloride/sodium citrate (SSC) at 22° C.
(c) sequences which are homologous to the nucleic acid sequences of group (a), wherein at least 90% of said sequences are identical to homologous sequence of group (a).

48. A collection according to claim 47 wherein said nucleic acid molecules are located in one or more arrays on a substrate.

49. A collection according to claim 47 comprising at least about 10,000 non-identical nucleic acid molecules.

50. A collection according to claim 47 comprising at least about 15,000 non-identical nucleic acid molecules.

51. A collection according to claim 47 comprising at least about 20,000 non-identical nucleic acid molecules.

52. A collection according to claim 47 comprising at least about 30,000 non-identical nucleic acid molecules.

53. A method for determining gene expression comprising
(a) collecting mRNA from tissue of an organism;
(b) using said mRNA as a template for producing a quantity of a labeled nucleic acid molecule;
(c) contacting said labeled nucleic acid molecule with a collection of purified nucleic acid molecules according to claim 47.

54. A method according to claim 53, wherein said purified nucleic acid molecules are capable of said determining gene expression of at least 5000 Arabidopsis thaliana genes and said purified nucleic acid molecules are deposited in an array on a substrate.

55. A method according to claim 53, wherein said purified nucleic acid molecules are capable of said determining gene expression of at least 10,000 Arabidopsis thaliana genes and said purified nucleic acid molecules are deposited in an array on a substrate.

56. A method according to claim 53, wherein said purified nucleic acid molecules are capable of said determining gene expression of at least 15,000 Arabidopsis thaliana genes and said purified nucleic acid molecules are deposited in an array on a substrate.

57. A method according to claim 53, wherein said purified nucleic acid molecules are capable of said determining gene expression of at least 20,000 Arabidopsis thaliana genes and said purified nucleic acid molecules are deposited in an array on a substrate.
